(19) United States
(12) Patent Application Publication
(10) Pub. No.: US 2013/0317335 A1
(43) Pub. Date: Nov. 28, 2013

(54) Applanation Tonometry Apparatus and Case for Transport and Use Thereof

(71) Applicant: EYELAB GROUP, LLC, (US)

(72) Inventors: Bruce E. Cohan, Ann Arbor, MI (US); John T. Savina, Whitmore Lake, MI (US); Zvi Flanders, Ann Arbor, MI (US)

(73) Assignee: EYELAB GROUP, LLC, Ann Arbor, MI (US)

(21) Appl. No.: 13/888,850

(22) Filed: May 7, 2013

Related U.S. Application Data
(60) Provisional application No. 61/643,505, filed on May 7, 2012.

(51) Int. Cl. A61B 19/02; A61B 3/16

(52) U.S. Cl. CPC A61B 19/0248; A61B 3/16
USPC 600/405; 206/305

(57) ABSTRACT

A case for transportation and use of an applanation tonometry apparatus includes a housing including a base and a wall structure extending upwardly therefrom, the housing sized to receive the applanation tonometry apparatus therein. A lid is pivotally connected to an upper rim of the wall structure, the lid having a closed position overlying the housing and an open position rotated away from the housing, the lid supporting the applanation tonometry apparatus in the open position. A handle is pivotally connected to the lid and has a stored position within the lid and a use position rotated away from the lid, the handle supporting the lid in the open position by engagement with a support surface.

### CROSS-REFERENCE TO RELATED APPLICATIONS

This application claims the benefit of U.S. provisional Application No. 61/643,505 filed May 7, 2012, the disclosure of which is incorporated in its entirety by reference herein.

### TECHNICAL FIELD

Embodiments relate to an applanation tonometry apparatus for the measurement of intraocular pressure, and a case for the transport and use of the apparatus.

### BACKGROUND

Intraocular pressure (IOP) is a physiological parameter routinely measured by eye care professionals. Elevated IOP is the most important risk factor in primary open angle glaucoma (POAG) which, combined with normal tension glaucoma (NTG), is the second leading cause of irreversible blindness in the United States. Patients with POAG and NTG have the same characteristic optic neuropathy (cupping) and visual field loss, but in NTG the IOPs have never been found to be elevated. Elevated IOP is also found in patients with ocular hypertension (OHT), but not the neuropathy or field changes. The only current treatment for POAG, NTG and OHT is reduction of IOP.

The instrument that is the reference standard for IOP measurement is the Goldmann applanation tonometer, used worldwide by ophthalmologists for over 50 years. This instrument functions to flatten part of the cornea to measure eye pressure, wherein the pressure within the eye is determined by how much force is needed to flatten the cornea.

Glaucoma management, which is so dependent on IOP, would benefit greatly by the acquisition of more IOP data. Essentially all IOP measurements are obtained on visits to the ophthalmologist's office—usually one measurement during typical office hours, and rarely more than one visit every two or three months. In glaucoma management, there is no parallel to the ubiquitous monitoring by diabetic patients of capillary blood glucose or by arterial hypertensive patients of blood pressure and heart rate. For these conditions, adjuncts in patient care increase the volume of measurements during clinic hours as well as extend the monitoring beyond the eight hours that the clinic is open.

Measurement of IOP at different times of the day usually yields different readings, sometimes highest at night. However, there is considerable variability in the diurnal pattern between individuals. Differences in IOP throughout the day are of special interest. In some POAG patients, despite treatment which results in normal IOPs (measured in the ophthalmologist's office), cupping and field loss can progress. In NTG, cupping occurs and can progress in the presence of IOP within the normal statistical limits (measured in the ophthalmologist's office). In OHT, over time, cupping and field loss can develop. The question in these cases is whether the progression (in POAG and NTG) and development (in OHT) of glaucoma damage is due to elevated IOP at times of the day when they cannot be measured in the ophthalmologist's office.

The disadvantages for both patients and medical personnel of an institutional site in measuring diurnal IOP led to the idea of home tonometry, which Posner noted in 1965, having patients use a Maklakoff type tonometer (Eye & Ear Nose Throat Mon 1965, 44: 64-66). Jensen and Maumenee (Am J Ophthalmol 1976, 76: 929-932) and later Alpar (Glaucoma 1983, 5: 130-132) had a family member measure the patient's IOP with the Schiotz tonometer.

A more recent approach to measuring diurnal IOP in the home environment introduced the concept of self-tonometry. Two technically sophisticated instruments, both hand-held and based on the applanation principle of the Goldmann tonometer, have been studied. In Zeimer and Wilenski's instrument (IEEE Trans Biomed Eng 1982, 29: 178-183), the IOP endpoint is detected by a photodiode array optical device instead of the signature pattern recognition used in Goldmann tonometry. Draeger and group used a microprocessor controlled optical sensor (see Groenhoff et al., Int Ophthalmol 1992, 16: 299-303). Both showed promise in the hands of their inventors, but others have found the correlation of patient measurements and ophthalmologist measurements using the Goldmann tonometer problematic, and also found that these devices can be moderately difficult to use. What may be most significant is the limited interest in these instruments since their invention in the 1980's, despite the concurrent heightened awareness of the potential importance of diurnal IOP.

As would be expected, self-tonometry with non-contact tonometers (see Stewart et al., Ann Ophthalmol 1991, 23: 177-182; Carenini et al., Int Ophthalmol 1992, 16: 295-297) that have been shown to be less reliable than the Goldmann method in the hands of ophthalmologists has met with a general lack of professional interest. Finally, the Tono-Pen®, based on the McKay-Marg applanation principle, is used by some ophthalmologists' technicians for IOP screening. While it has occasionally been used for self-tonometry (see Kupin et al., Am J Ophthalmol 1993, 116: 643-644), it is not easy to apply to oneself, and an ophthalmologist would not depend on measurements with a screening instrument as a basis for clinical decisions.

### SUMMARY

In one embodiment, a case for transportation and use of an applanation tonometry apparatus includes a housing including a base and a wall structure extending upwardly therefrom, the housing sized to receive the applanation tonometry apparatus therein. A lid is pivotally connected to an upper rim of the wall structure, the lid having a closed position overlying the housing and an open position rotated away from the housing, the lid supporting the applanation tonometry apparatus in the open position. A handle is pivotally connected to the lid and has a stored position within the lid and a use position rotated away from the lid, the handle supporting the lid in the open position by engagement with a support surface.

In another embodiment, a case for transportation and use of an applanation tonometry apparatus includes a housing including a base and a wall structure extending upwardly therefrom, the housing sized to receive the applanation tonometry apparatus therein. A lid is pivotally connected to an upper rim of the wall structure, the lid having a closed position overlying the housing and an open position rotated away from the housing approximately 180 degrees from the closed position, the lid supporting the applanation tonometry apparatus in the open position. A handle is pivotally connected to an outer surface of the lid and has a stored position within the lid and a use position rotated away from the lid, the handle including telescoping legs for supporting the lid in the open position by engagement with a support surface.

In one embodiment, an applanation tonometry system includes a housing including a base and a wall structure extending upwardly therefrom, and an applanation tonometry apparatus disposed within the housing, the apparatus having a base plate. A lid is pivotally connected to an upper rim of the wall structure, the lid having a closed position overlying the housing and an open position rotated away from the housing. A handle is pivotally connected to the lid and having a stored position within the lid and a use position rotated away from the lid, the handle supporting the lid in the open position by engagement with a support surface. For use, the apparatus is raised vertically from the housing and rotated approximately 180 degrees in a generally horizontal plane to align with the lid in the open position, the lid supporting the applanation tonometry apparatus in the open position.

### BRIEF DESCRIPTION OF THE DRAWINGS

FIG. 1 is a perspective view of an applanation tonometry apparatus according to an embodiment;

FIG. 2 is a fragmentary, end elevational view of the apparatus of FIG. 1;

FIG. 3 is a schematic illustration of the correct applanation endpoint pattern for measurement of IOP;

FIG. 4 is a front perspective view of an applanation tonometry apparatus according to another embodiment;

FIG. 5 is a rear side elevational view of the apparatus of FIG. 4 arranged for testing of a patient's right eye by a caretaker;

FIG. 6 is a rear side elevational view of the apparatus of FIG. 4 arranged for testing of a patient's left eye by a caretaker;

FIG. 7 is a front side elevational view of the apparatus of FIG. 4 arranged for self-tonometry of a patient's right eye;

FIG. 8 is a front side elevational view of the apparatus of FIG. 4 arranged for self-tonometry of a patient's left eye;

FIG. 9 is a rear perspective view of the apparatus of FIG. 4;

FIG. 10 is a perspective view of a wheeled case for transportation and use of an applanation tonometry apparatus;

FIG. 11 is a perspective view of the case of FIG. 10 with the handle shown in an outward position;

FIG. 12 is a perspective view of the case of FIG. 10 with a lid shown in an open position;

FIG. 13 is a perspective view of the case of FIG. 10, with the handle in use as legs supporting the open lid;

FIG. 14 is a perspective view of the case of FIG. 10, with the apparatus raised to an appropriate height for use;

FIG. 15 is a perspective view of the case of FIG. 10, with the apparatus rotated to a position for use;

FIG. 16 is a perspective view of the case of FIG. 10, with a support stand from the lid engaging the apparatus;

FIG. 17 is a perspective view of a travel case for transportation and use of an applanation tonometry apparatus;

FIG. 18 is a perspective view of an embodiment of an applanation tonometry apparatus for use with the travel case of FIG. 17, wherein the forehead rest is shown in a folded position for storage;

FIG. 19 is a perspective view of the apparatus of FIG. 18 with the forehead rest shown in an upright position for use;

FIG. 20 is a perspective view of the apparatus of FIG. 18 disposed on the case of FIG. 17 for use; and

FIG. 21 is a schematic representation of the positioning of a patient's head with respect to the apparatus and case configuration of FIG. 20.

### DETAILED DESCRIPTION

As required, detailed embodiments of the present invention are disclosed herein; however, it is to be understood that the disclosed embodiments are merely exemplary of the invention that may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present invention.

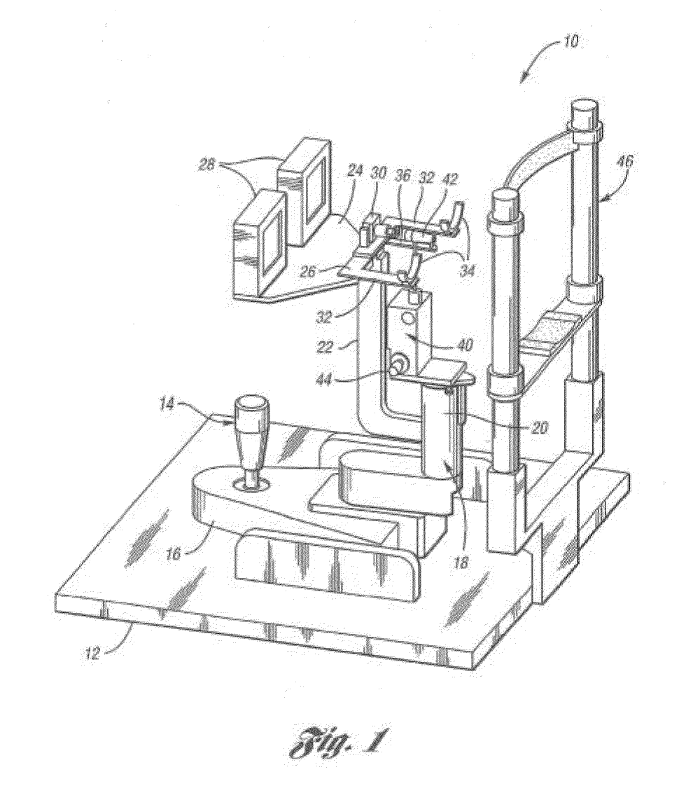

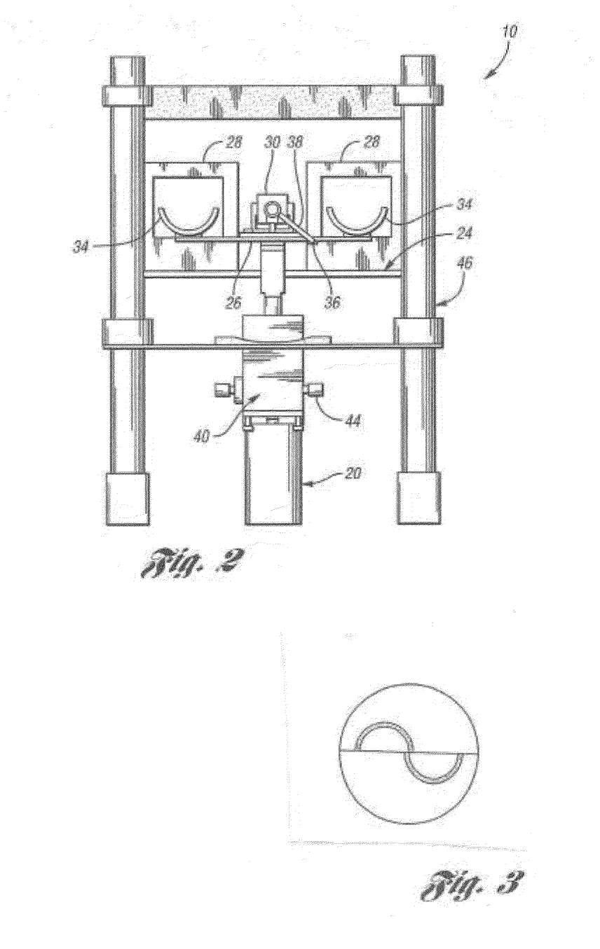

Embodiments are directed to an applanation tonometry apparatus for measurement of intraocular pressure (IOP). Referring first to FIGS. 1 and 2, a first embodiment of an applanation tonometry apparatus 10 is illustrated. Apparatus 10 was described in U.S. Pat. No. 7,556,380, incorporated by reference herein in its entirety. Apparatus 10 somewhat resembles a conventional slit lamp (Haag-Streit or the like) except that the microscope and illumination tower of the slit lamp are replaced with illumination and imaging components. Apparatus 10 comprises a housing which includes a base 12 with a joystick 14 and guide plate 16 disposed thereon, similar to a conventional slit lamp. Joystick 14 allows movement of guide plate 16 relative to base 12 in left and right directions, and toward and away from the user. The user can also turn the joystick 14 in a clockwise manner and in a counterclockwise manner to move guide plate 16 up and down. As best shown in FIG. 1, the housing further includes a support 18 that extends upwardly from guide plate 16 and includes a first post 20 for mounting a tonometer 40 and a second post 22 having mounting plates 24, 26 for mounting the various illumination and imaging components described below.

With continuing reference to FIGS. 1 and 2, mounting plate 24 is arranged to hold at least one display, such as two spaced LCD color video monitors 28. Mounting plate 26 is arranged to receive an ultra-miniature color video camera 30 which receives the applanation pattern and provides video output to monitors 28. Mounting plate 26 also includes first arms 32 which are provided with loose lens holders 34 attached thereto for receiving corrective lenses, both for refractive errors and presbyopia, if desired to accommodate the approximately 6 inch distance between a user's eye and video monitors 28. Mounting plate 26 also includes a second arm 36 arranged to hold an illuminator 38, typically a blue LED. A tonometer 40, preferably a standard Goldmann applanation tonometer, having a tonometer tip 42 and tonometer dial 44 is attached to first post 20 in alignment with video camera 30 in order to image the applanation pattern as described below. As best shown in FIG. 2, illuminator 38 is placed adjacent to tonometer tip 42, wherein the angle of illuminator 38 shown herein is merely exemplary. A chin-forehead rest 46 is also attached to base 12 as in a conventional slit lamp.

The wiring for each component described above preferably runs to a single cable connector and then to a power supply (not shown), wherein the wiring is preferably minimized and the transformers simplified for home use. Apparatus 10 is designed for portability in that it is light, compact and easy to use in a user's home environment, or other location outside of a physician's office. Lightweight materials, such as aluminum and plastics, can be used to construct base 12, joystick 14 and guide plate 16, support 18, and chin-forehead rest 46, and compactness can be achieved with telescoping vertical supports of chin/forehead rest 46. Apparatus 10 can be used with ease for testing the IOP of either the left or right eye.

The adjustment of tonometer tip 42 could be automated to obtain the endpoint applanation pattern. Proximity devices could be used to detect the presence of the eye as the tonometer tip 42 is applied to the cornea. Once the tip 42 is in contact with the cornea, image recognition software could use stepper motors to move the tonometer 40 through its 3-axes of movement (up and down, right and left, toward and away) until the applanation pattern is centered and the endpoint pattern is reached.

Additionally, external monitoring can be accomplished by viewing output from video camera 30 on an additional video monitor. The applanation pattern image being viewed by the user can then be simultaneously viewed by the physician, and is helpful for teaching users how to use the apparatus 10. Self-tonometry data can also be recorded to provide a direct, valid, verifiable, highly dependable assessment of the reliability of use of the apparatus 10 at home or another location outside of the office. A video recorder can be provided in communication with the video camera 30, and the output of a microprocessor analyzed to provide the applanation pattern images and the IOP readings, respectively, for subsequent assessment of the applanation endpoint patterns users obtain at home. The recording could be activated by the user's pressure on sensors (not shown) provided in the chin-forehead rest 46. Data recording during self-tonometry could include the day, time, a still image of the applanation endpoint pattern, and the IOP.

Prior to initiating testing using apparatus 10 described above, dye (for example, fluorescein) and anesthetic (for example, benoxinate, Fluorox, Ocusoft, Inc.) drops may be instilled in the user's test eye. The dye allows for easier viewing of the tear meniscus between the cornea and the tonometer tip 42, and the anesthetic numbs the surface of the eye to ensure that the user does not feel any discomfort during testing.

When using apparatus 10, the user takes position in contact with chin-forehead rest 46, and moves the joystick 14 with one hand for course positioning to bring the tonometer tip 42 close to the test eye, aligning the tip 42 by looking directly at it such that it appears symmetric. Using the joystick 14, the user then brings the tonometer tip 42 into contact with his/her cornea. As the tonometer tip 42 applanates (flattens) the cornea of the test eye, the user views the applanation pattern (typically green in color) on one video monitor 28 with the observing eye. Next, the user manipulates the joystick 14 to adjust the tonometer tip 42 position to center the applanation pattern on the monitor 28. Finally, with the other hand, the user manipulates the tonometer dial 44 to obtain the applanation endpoint pattern for IOP measurement as described below. The user will then remove the tonometer tip 42 from the cornea using the joystick 14 and repeat the procedure on the other eye. FIG. 3 shows a schematic illustration of the correct applanation endpoint pattern for measurement of IOP. As shown, the half circles are centered and are the same size, and the inner edges of the half circles just meet.

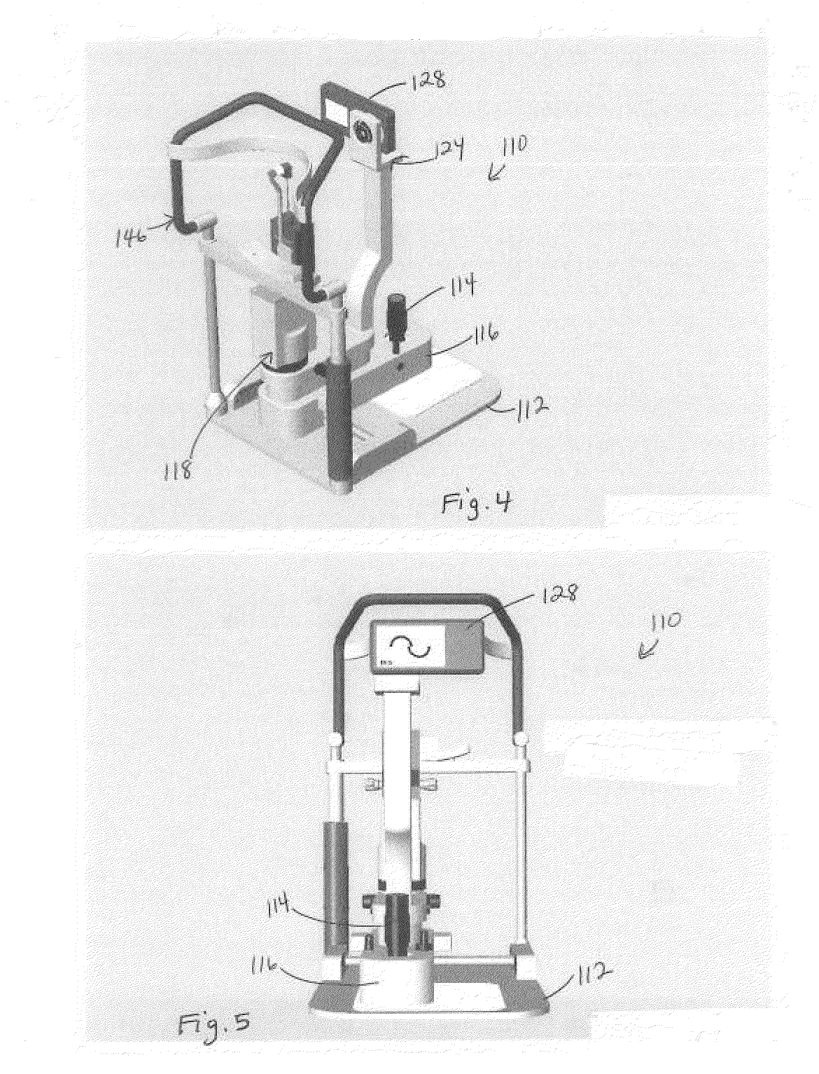

In addition to self-tonometry, the applanation tonometry apparatus can be configured to enable tonometry by another, such as a caretaker. In the embodiment depicted in FIGS. 4-9, a video monitor 128 which includes displays facing away from the patient as well as facing toward the patient is provided. It is understood that any of the features and components of apparatus 10 may also be applicable to apparatus 110 shown in FIGS. 4-9, wherein like components are given like reference numerals with the addition of a “1” prefix.

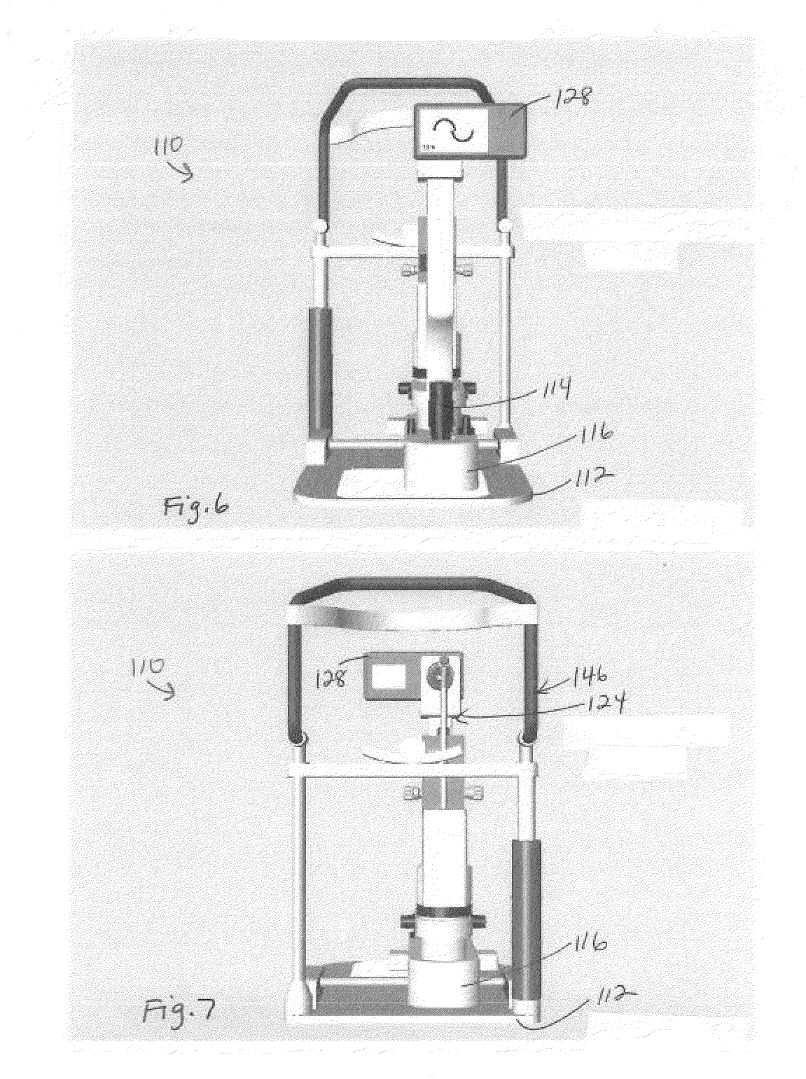

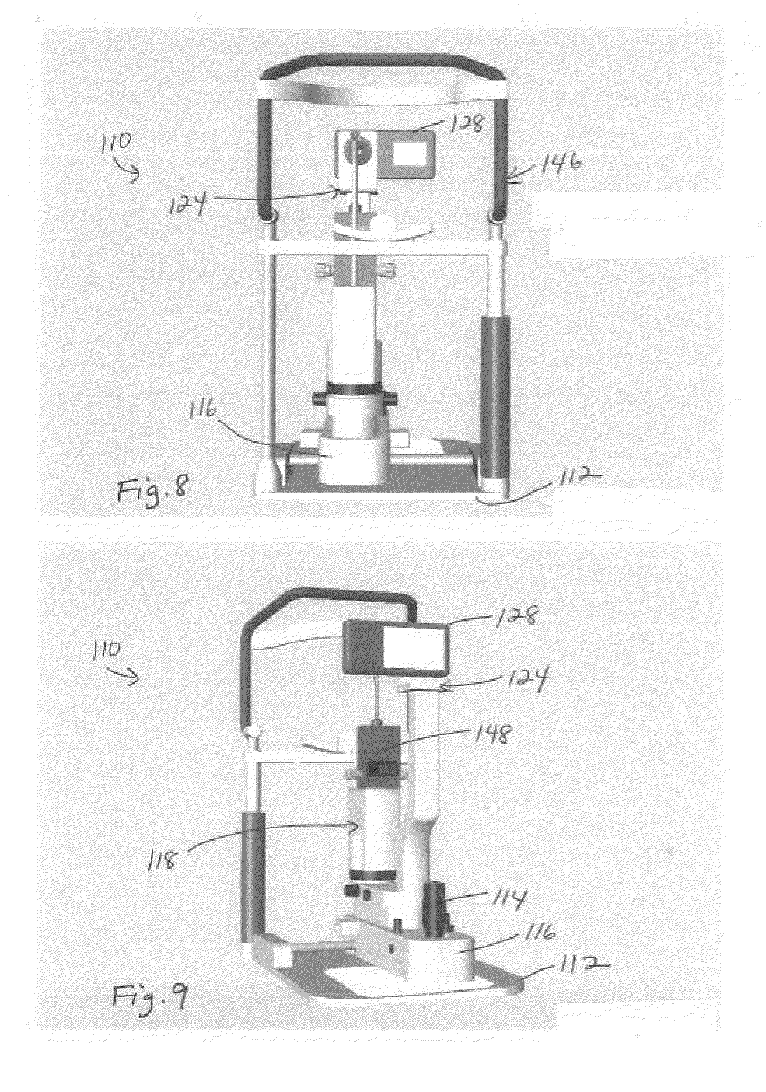

FIG. 4 shows a front side perspective view of the apparatus 110 positioned for self-tonometry, while FIG. 9 shows a rear side perspective view of the apparatus 110 positioned for tonometry by a caretaker. In FIG. 9, a display screen 148 is illustrated for providing an indication of IOP. FIGS. 5 and 6 illustrate a position of the video monitor 128 and apparatus 110 for tonometry of the right eye and left eye of a patient, respectively, by a caretaker. FIGS. 7 and 8 illustrate a position of the video monitor 128 and apparatus 110 for self-tonometry of the right eye and left eye of a patient, respectively. In this embodiment, the video monitor 128 or other display is pivotally mounted on the mounting plate 124, such that it can be rotated for proper positioning when moving between measurements of the right and left eye. Alternately, dual monitors 128 could be provided.

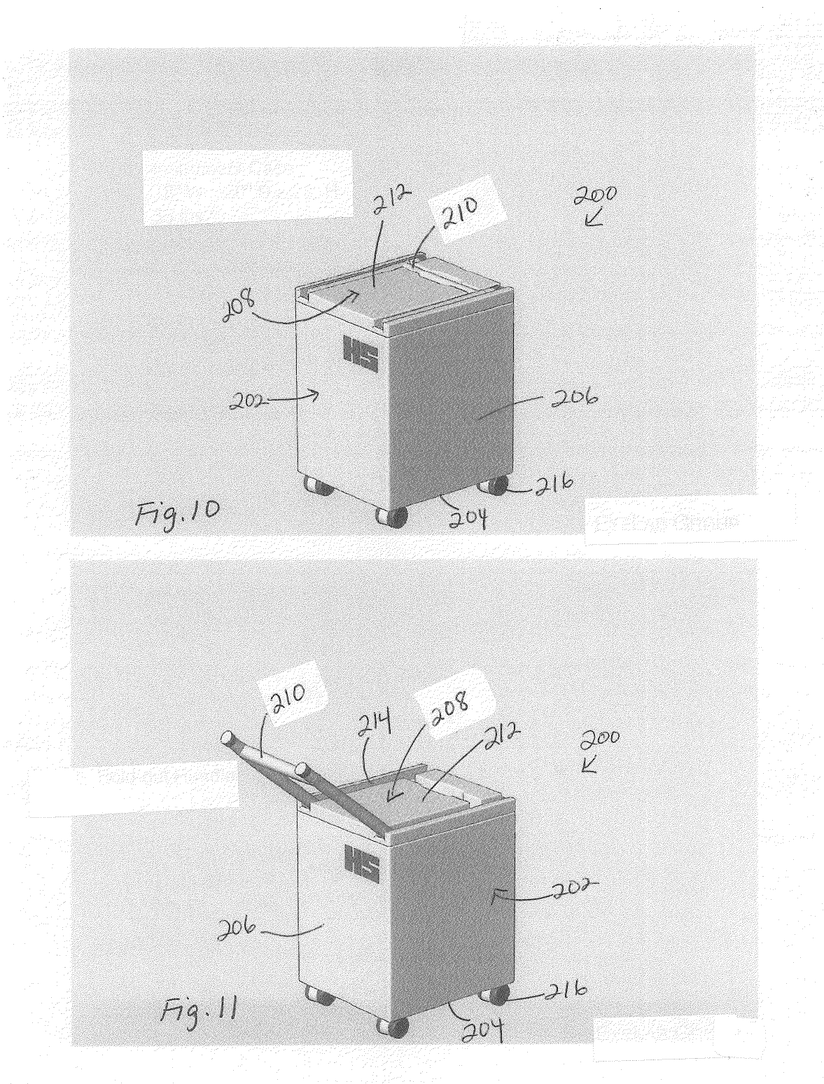

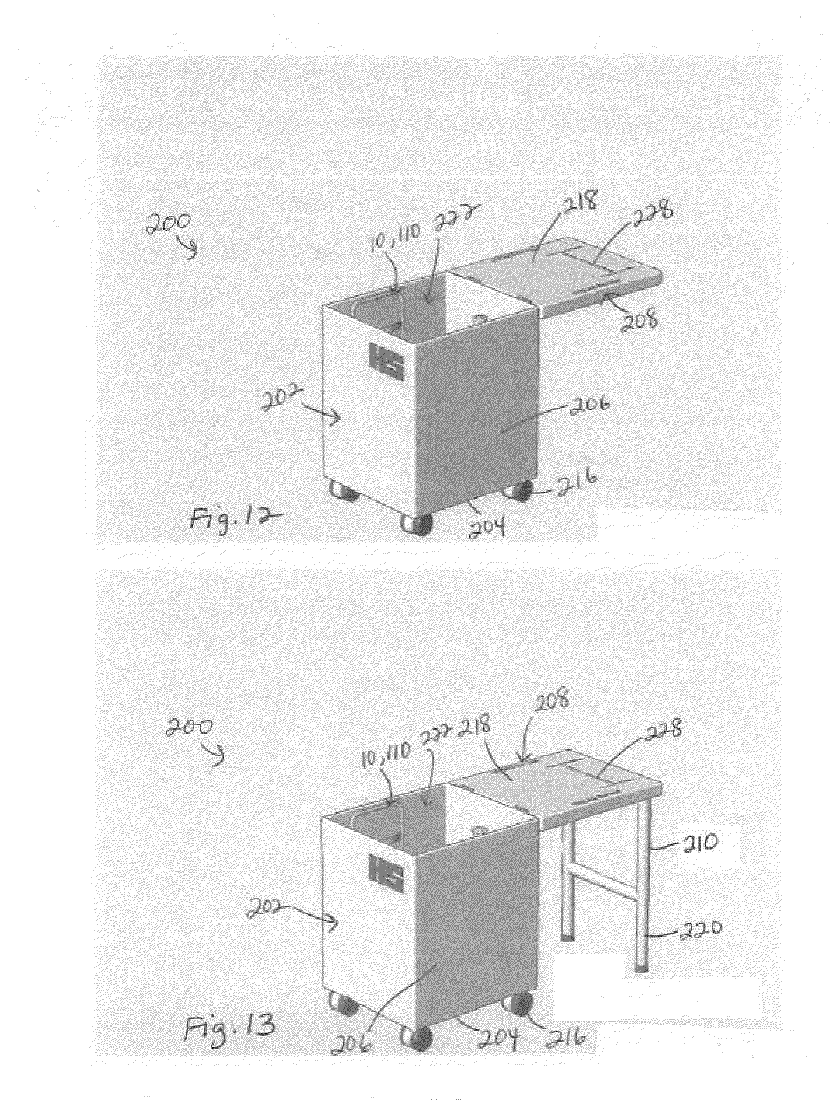

With reference to FIGS. 10-16, an embodiment of a case 200 for transportation and use of an applanation tonometry apparatus (such as apparatus 10 or apparatus 110, but not limited thereto) is illustrated. As shown in FIG. 10, the case 200 has the general appearance of a piece of luggage or a salesperson's case, and may comprise a box-like housing 202 having a base 204 and a wall structure 206 extending upwardly from the base 204. In accordance with one non-limiting embodiment, the case 200 may have dimensions of approximately 18″ W×20″ D×28″ H and weigh approximately 35 lbs. In one embodiment, a lid 208 is pivotally connected to an upper rim of the wall structure 206, and a handle 210 is pivotally connected to a first, outer surface 212 of the lid 208. Lid outer surface 212 may include a recess 214 to allow the handle 210 to nest therewithin in a stored position when not in use. FIG. 11 illustrates the rotation of the handle 210 to a use position away from the lid 208 for use in pushing or pulling the case 200. The case 200 may include wheels 216 to allow it to be wheeled to the site where the apparatus is to be used, and a brake (not shown) may be provided for locking the wheels 216 once the case 200 is in its desired location. As shown in FIG. 12, the hinged lid 208 may be opened from a closed position overlying the housing 202 into an open position approximately 180 degrees from the closed position. The open position may be parallel with the floor or other support surface, such that the lid inner surface 218 when closed is now the top surface. As depicted in FIG. 13, the handle 210 becomes a lid support assembly, as the handle 202 is rotated downward from the lid 206 into a generally vertical position. In one embodiment, the handle 210 includes telescoping legs 220 that are extended until they meet the floor or other support surface to allow for adjustable engagement.

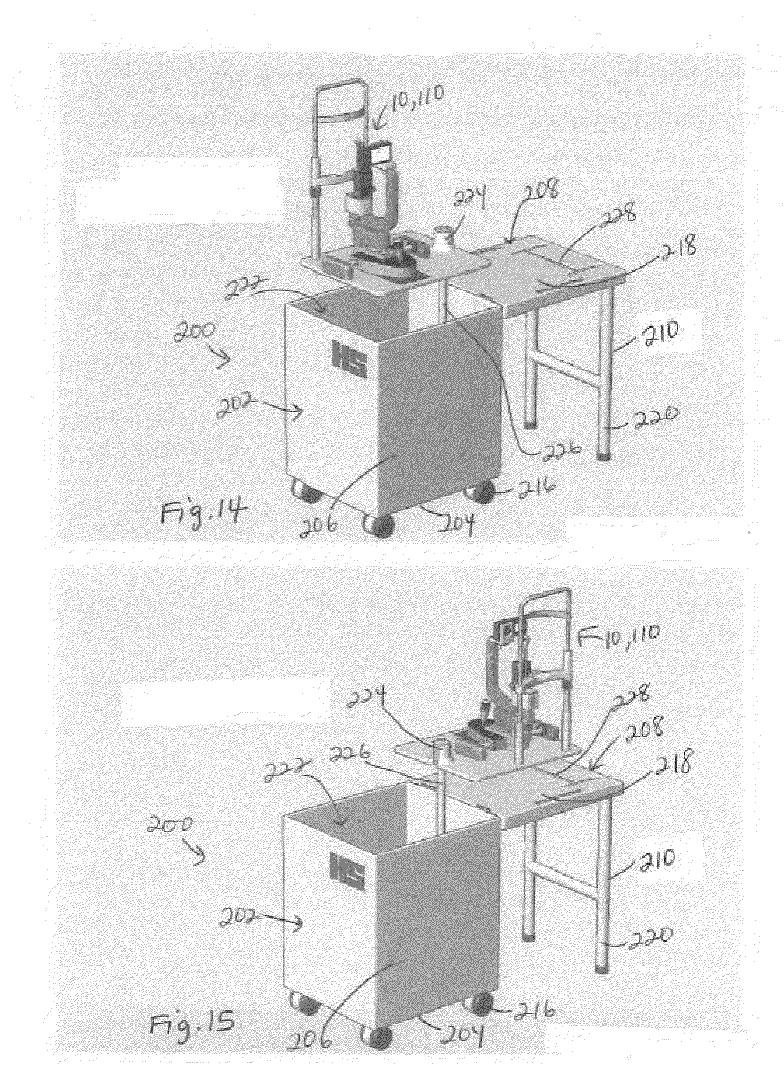

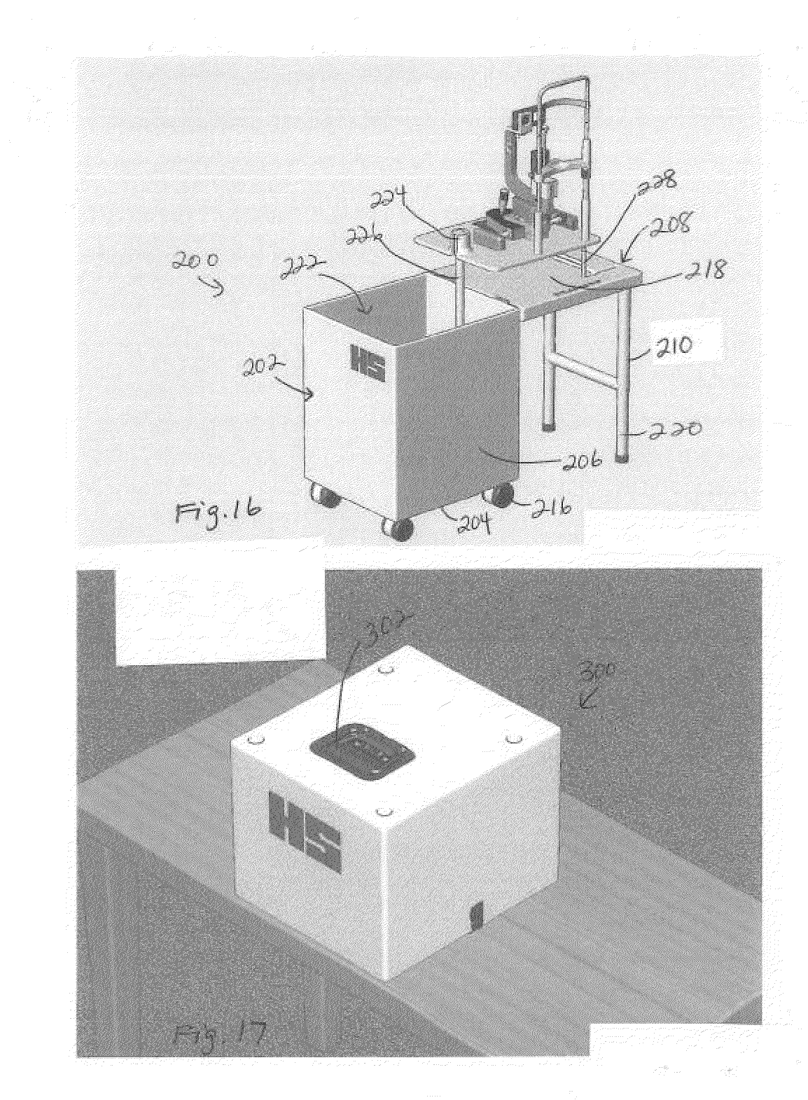

With reference to FIG. 14, the apparatus 10, 110 may be raised manually by the patient or caregiver from the case interior 222, or alternatively could be raised by a powered (e.g., pneumatic) mechanism. The apparatus may be guided by a bushing 224 along a rod 226 of a lift/support assembly, until it engages a latching feature (not shown), locking it in place. As shown in FIG. 15, the apparatus 10, 110 may then be rotated approximately 180 degrees in a generally horizontal plane until it reaches a hard-stop, positioning it over the open lid 208 for use. A hinged support stand 228 may be rotated upward from a nested storage position within the lid inner surface 218, as shown in FIG. 16, engaging a bottom surface of the apparatus base plate.

FIGS. 17-21 illustrate an embodiment of a travel case 300 for transportation and use of an applanation tonometry apparatus. It is understood that features and components of the apparatuses 10, 110 and case 200 described previously herein may also be applicable to the apparatus 210 and travel case 300 shown in FIGS. 17-21. For apparatus 210, like components with apparatus 10 and/or apparatus 110 are given like reference numerals with the substitution of a “2” prefix.

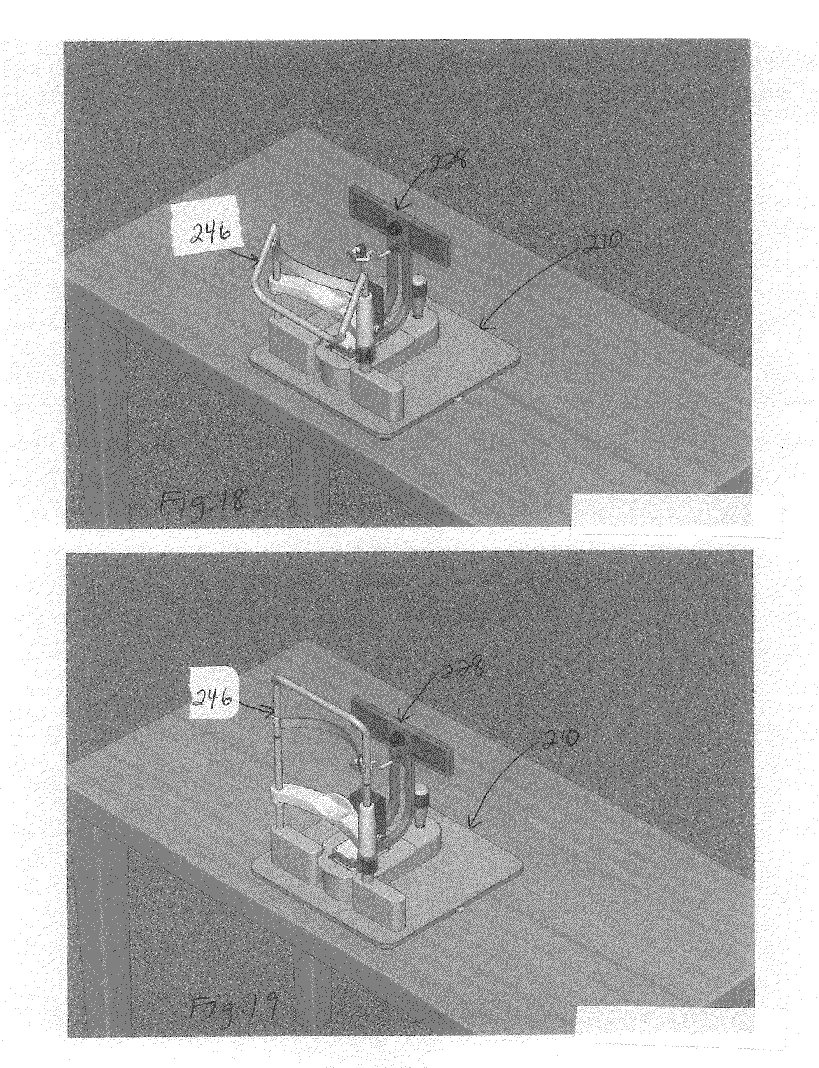

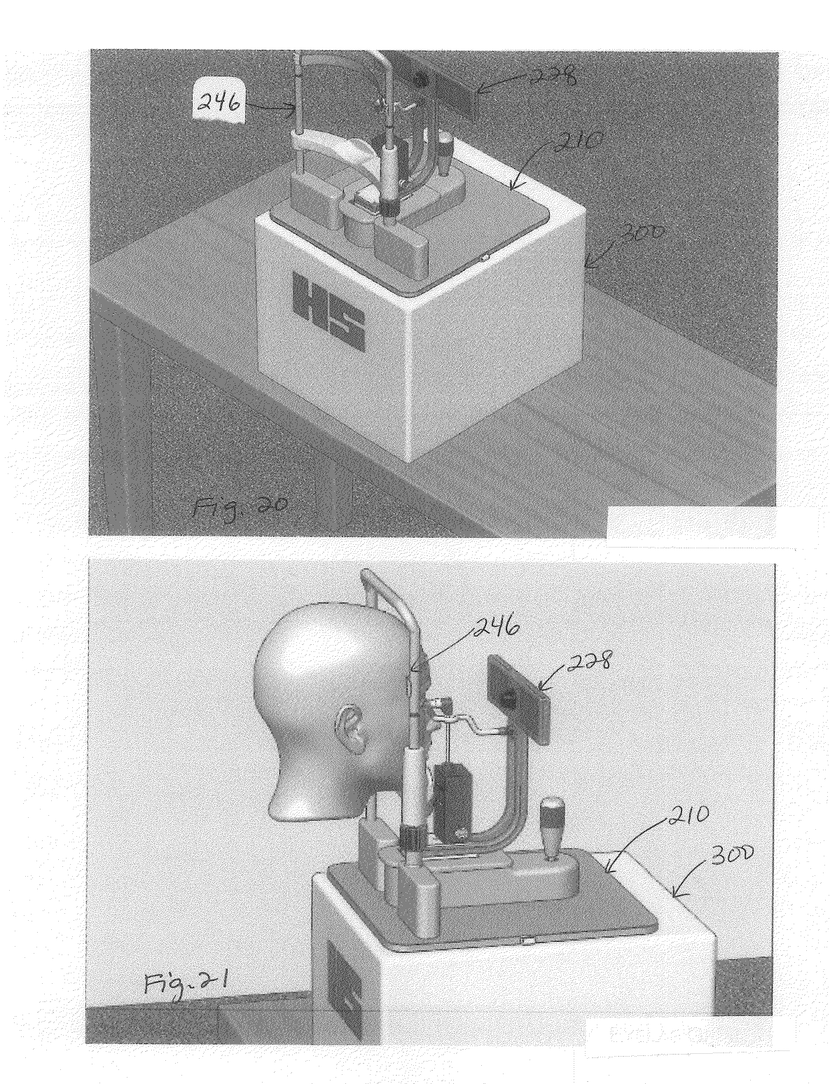

FIG. 17 depicts a perspective view of an embodiment of a travel case 300 on an elevated support surface, such as a table. In accordance with one non-limiting embodiment, the case 300 may have dimensions of approximately 12″ H×12″ W×14″ D and weigh approximately 12 lbs. The case 300 may be provided with a handle 302 for ease of transportation, and recesses for receiving a base portion of the apparatus 210 when in use. FIG. 18 is a perspective view of an embodiment of an applanation tonometry apparatus 210 for use with the travel case of FIG. 17, wherein a hinged or otherwise movable forehead rest 246 is shown in a folded position for storage, while FIG. 19 depicts the forehead rest 246 shown in an upright position for use. FIG. 20 is a perspective view of the apparatus 210 disposed on the case 300 for use, and FIG. 21 is a schematic representation of the positioning of a patient's head with respect to the apparatus 210 and case 300 configuration. The overall height of the apparatus 210 may be reduced in this embodiment since proper vertical positioning is provided by placement on top of the case 300.

The apparatus described herein provides a diagnostic tool that will enable ophthalmologists to obtain a vastly increased volume of user IOP information throughout 24 hours. This could greatly improve medical control of IOP, the primary risk factor of glaucoma.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. A case for transportation and use of an applanation tonometry apparatus, comprising:
a housing including a base and a wall structure extending upwardly therefrom, the housing sized to receive the applanation tonometry apparatus therein;
a lid pivotally connected to an upper rim of the wall structure, the lid having a closed position overlying the housing and an open position rotated away from the housing, the lid supporting the applanation tonometry apparatus in the open position; and
a handle pivotally connected to the lid and having a stored position within the lid and a use position rotated away from the lid, the handle supporting the lid in the open position by engagement with a support surface.

2. The case of claim 1, wherein the handle is pivotally connected to an outer surface of the lid.

3. The case of claim 2, wherein the lid outer surface includes a recess that receives the handle.

4. The case of claim 1, wherein the open position of the lid is approximately 180 degrees from the closed position.

5. The case of claim 1, wherein the handle includes telescoping legs for adjustably engaging the support surface.

6. The case of claim 1, further comprising a plurality of wheels attached to the base.

7. The case of claim 1, further comprising a vertical rod disposed within the housing and extending through a base plate of the applanation tonometry apparatus.

8. The case of claim 7, further comprising a bushing formed in the base plate to guide the applanation tonometry apparatus along the rod.

9. The case of claim 1, further comprising a support stand pivotally connected to an inner surface of the lid, the support stand rotatable to engage a base plate of the applanation tonometry apparatus.

10. A case for transportation and use of an applanation tonometry apparatus, comprising:
a housing including a base and a wall structure extending upwardly therefrom, the housing sized to receive the applanation tonometry apparatus therein;
a lid pivotally connected to an upper rim of the wall structure, the lid having a closed position overlying the housing and an open position rotated away from the housing approximately 180 degrees from the closed position, the lid supporting the applanation tonometry apparatus in the open position; and
a handle pivotally connected to an outer surface of the lid and having a stored position within the lid and a use position rotated away from the lid, the handle including telescoping legs for supporting the lid in the open position by engagement with a support surface.

11. An applanation tonometry system, comprising:
a housing including a base and a wall structure extending upwardly therefrom;
an applanation tonometry apparatus disposed within the housing, the apparatus having a base plate;
a lid pivotally connected to an upper rim of the wall structure, the lid having a closed position overlying the housing and an open position rotated away from the housing; and
a handle pivotally connected to the lid and having a stored position within the lid and a use position rotated away from the lid, the handle supporting the lid in the open position by engagement with a support surface,
wherein for use the apparatus is raised vertically from the housing and rotated approximately 180 degrees in a generally horizontal plane to align with the lid in the open position, the lid supporting the applanation tonometry apparatus in the open position.

12. The system of claim 11, wherein the handle is pivotally connected to an outer surface of the lid.

13. The system of claim 12, wherein the lid outer surface includes a recess that receives the handle.

14. The system of claim 11, wherein the open position of the lid is approximately 180 degrees from the closed position.

15. The system of claim 11, wherein the handle includes telescoping legs for adjustably engaging the support surface.

16. The system of claim 11, further comprising a plurality of wheels attached to the base.

17. The system of claim 11, further comprising a vertical rod disposed within the housing and extending through the apparatus base plate.

18. The system of claim 17, further comprising a bushing formed in the apparatus base plate to guide the apparatus along the rod.

19. The system of claim 11, further comprising a support stand pivotally connected to an inner surface of the lid, the support stand rotatable to engage a bottom surface of the apparatus base plate.
